(19) United States
(12) Patent Application Publication
(10) Pub. No.: US 2018/0313715 A1
(43) Pub. Date: Nov. 1, 2018

(54) PIPELINE INSPECTION ROBOT

(71) Applicant: NATIONAL GRID GAS PLC, LONDON (GB)

(72) Inventors: RAFAL CICHOSZ, HARROGATE (GB); JOHN WHITE, LEEDS (GB); TOM PRICE, YORK (GB); CHRIS BARKER, LEEDS (GB)

(21) Appl. No.: 15/777,632

(22) PCT Filed: Nov. 16, 2016
(86) PCT No.: PCT/EP2016/077861

§ 371 Date: May 18, 2018

(30) Foreign Application Priority Data
Nov. 20, 2015 (GB) 1520462.1

(51) Int. Cl. G01M 3/00; F16L 55/32; G01M 3/40; G01N 33/00; G01N 21/3504; G01N 29/12; B25J 5/00; B62D 55/02; B60B 33/00; B60B 33/08; B60B 19/00; G05D 1/02; H04N 7/18; G06K 9/00

(52) U.S. Cl. CPC G01M 3/005; F16L 2101/30; G01M 3/40; G01N 33/0047; G01N 21/3504; G01N 29/12; B25J 5/005; B62D 55/02; B60B 33/00; B60B 33/08; B60B 19/006; G05D 1/021; H04N 7/185; G06K 9/00664; F16L 55/32

(57) ABSTRACT

The present invention provides a robot which is suitable for travel through a pipeline. The inventive robot comprises at least one tracked drive means and at least one roller means that can swivel about an axis substantially normal to a rolling axis thereof, wherein said at least one tracked drive means and at least one roller means are provided with magnetic means for generating a magnetic adhesion force between the robot and an internal wall of the pipeline.

### TECHNICAL FIELD

The present invention relates to a mobile robot for internally inspecting pipelines, a robotic system comprising two or more such robots and to a method for pipeline inspection as defined in the present independent claims.

### BACKGROUND OF THE INVENTION

Typical pipe constructions such as sewers, gas/oil transmission pipelines, gas/oil distribution pipes are suffering from several diseases when getting old. (Note: A “pipeline” or “pipe” may alternatively be termed simply as “conduit”, and as used herein such terms may be used interchangeably). Aging, corrosion and mechanical stress generally lead to the loss of material thickness or generation of cracks that can cause leakages or sometimes the destruction of the pipeline construction

Thus, periodic inspection of the pipe system is required in order to prevent such damages. Since many of these constructions have not been designed to optimize automatic inspection and repair tasks, inspection and maintenance generate huge costs, especially if disassembling or even excavating is necessary. Inspection and maintenance technology has then become a growing industry and a large variety of systems have been developed.

For the maintenance of pipelines devices known as “pigs” are used often. “Pig” is sometimes claimed as an acronym or backronym derived from the initial letters of the term “pipeline inspection gauge” or “pipeline intervention gadget”. Accordingly, “pigging” in the context of pipelines refers to the practice of using “pigs” to perform various maintenance operations on a pipeline. This is done without stopping the flow of the product in the pipeline. These operations include but are not limited to cleaning and inspecting the pipeline. This is accomplished by inserting the pig into a “pig launcher” (or “launching station”)—an oversized section in the pipeline, reducing to the normal diameter. The launcher is then closed and the pressure-driven flow of the product in the pipeline is used to push the pig along down the pipe until it reaches the receiving trap—the “pig catcher” (or “receiving station”).

Such “pigs” are often designed to be as small and lightweight as possible, and to that end it is common practice to provide such apparatuses or “robots” with a multi-strand or multi-tube tether or umbilical cable. An umbilical cable is a cable that allows the robot to be provided in communication with an external body (such as an above-ground control station). The cable may for example enable the robot to be in electrical communication (allowing data signals to be exchanged and/or electrical power to be supplied), fluid communication (e.g. allowing fluid such as compressed gas, such as compressed air, or liquid such as water, to be provided to the robot) or optical communication (e.g. allowing optical data signals to be provided via a fibre-optic cable). It is named by analogy with an umbilical cord. The term “communication” in this sense refers to a connection. Such pigs or robots may also comprise several different sections or modules, e.g. each being constructed, designed and controlled to perform a given unique operation in the overall pipeline maintenance procedure.

However, some pipelines are “unpiggable”, for example because a free swimming tool can't be introduced or removed. Other barriers to pigging are insufficient flow to overcome friction and drive a pig, multi-diameters pipes or internal obstacles.

Some possible alternative locomotion strategies used to solve the in-pipe inspection problems are robots of the wheel type, caterpillar type, wall-pressed type, walking type, inch-worm type or screw type. Bends of the pipe are usually overcome thanks to differential-drive steering (for single body systems) or articulated structures.

When climbing ability is required, the most common solution is to use robots with spreading systems. However, none of these systems can deal with narrow pipe environments which integrate high abrupt diameter changes and bends and also require climbing ability. In this case, it has been tried to combine the locomotion system of the robots with attachment elements such as grasps, suction cups, adhesive polymers or (electro)magnetic elements. Since these concepts usually imply complex mechanics and since the considered environment is ferromagnetic magnetic attachment systems have been considered. These robots take advantage of the magnetic force in order to travel on surfaces with any inclination, however, their mobility is limited to smooth obstacle-free surfaces.

Accordingly, there is still a substantial need for mobile robots which are suitable to inspect complex shaped pipe structures. Such complex pipe structures may have a wide range of inner diameters and may be composed of horizontal and vertical pipe elements. In addition, internal obstacles such as bends and pipeline fittings (T-branches, Y-branches), and any inclination can be encountered.

It is an object of the present invention to provide a mobile robot for pipeline inspection that is capable to address disadvantages associated with the prior art such as discussed above.

### SUMMARY OF THE INVENTION

In one aspect the present invention provides a robot as defined in present independent claim 1 which is suitable for travel through a pipeline. The inventive robot comprises at least one tracked drive means and at least one roller means that can swivel about an axis substantially normal to a rolling axis thereof, wherein said at least one tracked drive means and at least one roller means are provided with magnetic means for generating a magnetic adhesion force between the robot and an internal wall of the pipeline.

In a further aspect the present invention provides a robot as defined in present independent claim 2 which is suitable for travel through a pipeline. The inventive robot comprises a body having a streamlined aerofoil shape form that promotes pressing of the robot to the internal wall of the pipeline.

In yet a further aspect the present invention provides a robot as defined in present independent claim 3 which is suitable for travel through a pipeline. The inventive robot comprises at least one tracked drive means and at least one roller means that can swivel about an axis substantially normal to a rolling axis thereof, wherein said at least one tracked drive means and at least one roller means are provided with magnetic means for generating a magnetic adhesion force between the robot and an internal wall of the pipeline; and wherein said robot comprises a body having a streamlined aerofoil shape form that promotes pressing of the robot to the internal wall of the pipeline.

By means of its magnetic locomotion or traction system the inventive robot of an embodiment of the present invention can travel through pipelines with any inclination regarding the gravity vector and can also easily climb into vertical pipeline segments. The magnetic adhesion force between the robot and the internal wall of the pipeline securely maintains the robot in contact with the surface. Internal obstacles such as bends or pipeline fittings (T-branches, Y-branches) are overcome with the help of the tracked drive means in a similar manner as a caterpillar is driving through impassable terrain. The speed of the tracked drive means and their motion or movement direction can be controlled independently, for example, one track rotates forwards and the other backwards, providing steering capability to go through 45° or 90° bends, T-branches and Y-branches.

In another aspect the present invention provides robotic systems as defined in present independent claims 21 and 22. In an embodiment of the inventive robot system said system comprises two robots as defined above, wherein said two robots are connected to each other in such a way that their at least one tracked drive means and at least one roller means are arranged opposite to each other and when said robotic system travels through a pipeline are efficiently pressed to the pipeline wall. This robotic system is therefore of the well-known wall-pressed type.

In an embodiment of the present invention, the inventive robots described above may be provided with a multi-strand or multi-tube tether or umbilical cable via which it is linked to an above-ground control station (for example a computer workstation) and sources of electrical power, operational control signals, supplies of pressurised fluid to onboard pneumatic and/or hydraulic systems, and suchlike. Instead of a tether or umbilical cable the communication with the control station may be wireless, e.g. radio, optical or acoustically.

In another embodiment of the inventive robot system said system comprises two or more (a “plurality”) robots as defined above. Said robots may be configured to cooperate with one another in such a way that when traveling through a pipeline they are distributed at different respective circumferential locations around the inner bore of the pipeline and move generally parallel to the axis of the pipeline. Optionally, the members of the robot group may be configured to communicate with one another. Optionally, a system of constant feedback between robots may be implemented that allows constant coordination (e.g. adjustment of direction/path of travel or speed) of individual robots in cooperation with others, as well as a change of the behavior of the whole group. The communication may be by only local communication, which, for example, can be achieved by wireless transmission systems. Such a robot system is more resistant to failure. Whereas one large robot may fail and ruin a mission, a group of robots can continue even if several robots fail.

The plurality of robots as defined above may each be configured to communicate with at least one other robot, optionally by a wireless communications link, optionally via a base station, alternatively or in addition directly with one another. The robots may be configured to cooperate to perform an inspection operation.

The inspection operation may comprise travel of the plurality of robots along the pipeline, each robot inspecting a respective area of the pipeline in a coordinated manner wherein substantially the whole of an interior surface of a predetermined length of pipeline is inspected.

In some arrangements, substantially the whole of the interior surface of the predetermined length of pipeline is inspected during the inspection operation. The inspection operation may comprise causing the plurality of robots to pass along the predetermined length of pipeline a predetermined number of times, which may be one, two, three or more times.

Each path followed by a robot over the predetermined length during the inspection operation may be substantially unique. Optionally, no robot follows the same path more than once during a given inspection operation. Further optionally no two robots follow substantially the same path during a given inspection operation.

Each time a robot travels along the predetermined length it may inspect a portion of the pipeline that is not inspected more than once during a given inspection operation.

The plurality of robots may be arranged to inspect the pipeline by means of one or more NDT sensor devices such as by means of acoustic resonance inspection, ultrasonic inspection, eddy current inspection, capture of one or more images such as video images or any other suitable method. The robots may be configured to detect the presence of one or more chemical species in some embodiments.

Each of the plurality of robots may comprise a body having a streamlined aerofoil shape form that promotes pressing of the robot to the internal wall of the pipeline.

In yet another aspect, the present invention provides a method for pipeline inspection as defined in present independent claim 23. The inventive method comprises moving at least one robot or robotic system as defined above along a pipeline within a pipeline network; inspecting said pipeline for leaks or failures using the at least one machine vison system and/or the at least one NDT device and/or the at least one other sensor device of said robot or robotic system; and tracking the position of said robot or robotic system within said pipeline using the at least one locating device, e.g. a global positioning system (GPS).

Other aspects, objects and advantages of the invention will be apparent from the following detailed description of exemplary or preferred embodiments considered in conjunction with the accompanying drawings of those exemplary or preferred embodiments.

Advantageous and/or preferred embodiments of the invention are subject matter of the respective sub-claims.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible, or unless it is otherwise indicated herein or otherwise clearly contradicted by context. The applicant reserves the right to change any originally filed claim or file any new claim accordingly, including the right to amend any originally filed claim to depend from and/or incorporate any feature of any other claim although not originally claimed in that manner.

The skilled artisan will appreciate that the use of the terms “one”, “a”, “an” and “the” and similar referents in the context of describing the invention (especially in the context of the following claims) is to be construed to cover both the singular and the plural, i.e. is intended to include “at least one” or “one or more,” unless otherwise indicated herein or clearly contradicted by context. The terms “comprising,” “having,” “including,” and “containing” are to be construed as open-ended terms (i.e., meaning “including, but not “limited to ”) unless otherwise noted. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless otherwise expressly specified, all of the numerical ranges, amounts, values and percentages, such as those for amounts of materials, elemental contents, times and temperatures, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention, At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements.

When percentages by weight are used herein, the numerical values reported are relative to the total weight.

Also, it should be understood that any numerical range recited herein is intended to include all sub-ranges subsumed therein. Recitation of ranges of values herein are merely intended to serve as a shorthand method of referring individually to each separate value falling within the range, unless otherwise indicated herein, and each separate value is incorporated into the specification as if it were individually recited herein. Furthermore, when numerical ranges are set forth herein, these ranges are inclusive of the recited range end points (i.e. end points may be used). For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Unless otherwise defined, all technical and scientific terms used herein have the same meaning as commonly understood by one of ordinary skill in the art to which this invention belongs. Although methods and materials similar or equivalent to those described herein are used in the practice or testing of the present invention, suitable methods and materials are described below. All publications, patent applications, patents, and other references mentioned herein are incorporated by reference in their entirety. In case of conflict, the present specification, including definitions, will control.

While the invention in the following will be described in connection with certain exemplary or preferred embodiments, including the best mode known to the inventors for carrying out the invention, there is no intent to limit it to those embodiments. Variations of those exemplary or preferred embodiments may become apparent to those of ordinary skill in the art upon reading the description. For example, each feature disclosed in this specification may be replaced by an alternative feature serving the same, equivalent, or similar purpose. Thus, unless expressly stated otherwise, each feature disclosed is only an example of a generic series of equivalent or similar features. The inventors expect skilled artisans to employ such variations as appropriate, and the inventors intend for the invention to be practiced otherwise than as specifically described herein. Accordingly, the invention includes all alternatives, modifications and equivalents of the subject matter recited in the claims appended hereto as permitted by applicable law.

In one embodiment of the invention, the magnetic roller means of the inventive robot are selected from the group consisting of castor (or caster) wheels and roller-balls wheels. Both types of wheels may be combined if suitable or desired. In general, a castor wheel is an undriven, single, double or compound wheel that is designed to be mounted to the bottom of a larger object (the “vehicle”) so as to enable that object to be easily moved. The castor wheels used in the invention is advantageously a swivel castor. A swivel castor incorporates a wheel mounted to a fork, wherein an additional swivel joint above the fork allows the fork to freely rotate about 360°, thus enabling the wheel to roll in any direction. This makes it possible to easily move the vehicle in any direction without changing it orientation. Roller-ball wheels comprise a roller ball that can freely rotate, for example in a roller-ball socket. The magnets used for the wheels may be permanent magnets or electromagnets. Permanent magnets (for example neodymium magnets) are preferred since they do not need electric current and have a lighter weight. Further, they are fail-safe. For example, the wheels may comprise ferromagnetic rings.

In another embodiment of the invention, the magnetic tracked drive means (or caterpillar tracks) of the inventive robot comprise a suspension system allowing the drive tracks to bend and keep constant traction and required tension. Suitable suspension systems are commercially available or may easily be adapted to the practical needs. The magnetic means of the tracked drive means may be the same as for the roller means. The shape of the tracks and suspension system is adequate to pipe curvature which will increase the contact area and maximise friction. In an embodiment the magnetic tracks are reinforced by metal dowel pins in each joint. The pins are locked in pockets which keep them in place.

In yet another embodiment of the invention, the inventive robot comprises a body having a streamlined aerofoil shape form that promotes pressing of the robot to the internal wall of the pipeline. An aerofoil (or airfoil) is the shape of a wing. An aerofoil-shaped body moved through a fluid produces an aerodynamic force which may be used for the invention's purpose to press the robot down. An example for an aerofoil shape found in nature is the dolphin flipper fin. The streamlined aerofoil shape used in the invention may be passive or active. Passive means that the aerofoil shape does not change in use, whereas active means that the aerofoil shape may change in response to external cues. Active aerofoil shapes may be made of an adaptive material, e.g. a magnetorheological elastomeric (MRE) material. MREs (also called magnetosensitive elastomers) are a class of solids that consist of polymeric matrix with embedded micro- or nano-sized ferromagnetic particles such as carbonyl iron. As a result of this composite microstructure, the mechanical properties of these materials can be controlled by the application of magnetic field. The body of the inventive robot may, for example, have a “monocoque” design or may be comprised of a chassis and a body carried by the chassis. A monocoque is a structural approach whereby loads are supported through an object's external skin, similar to an egg shell. If a chassis is used it do not need to have an aerofoil shape. It some pipeline environments it may be advantageous that the body (or chassis/body) is pressure-resistant and gas-tight.

In another embodiment of the invention, the inventive robot may comprise at least one machine vision (MV) system. MV is the technology and methods used to provide imaging-based automatic inspection and analysis for such applications as automatic inspection, process control, and robot guidance in industry. In the inventive robot the MV system may be mounted, for example, to the front or rear or to the front and rear of the robot. The MV system may comprise at least one video camera, for example, a fixed forwards facing camera, a self-levelling camera or a pan and tilt camera. The camera technology used may be digital or analogue. Analogue cameras are available in three main technologies each of which may be used in the invention: CMOS, CCD and CCIQ. These camera types are commercially available in a large selection. In some pipeline environments it may be advantageous that the camera is pressure-resistant and gas-tight. The video camera may operate in the visible or non-visible light spectrum. The non-visible light spectrum may be, for example, the infrared spectrum, the near-infrared spectrum or the ultra-violet spectrum. In addition the video camera may comprise at least one lighting array for illumination. The MV system allows the robot to recognise in pipe features and to self-center relative to the pipeline. It can also be used for location, guidance and as a check for tasks completed by the robot.

In another embodiment of the invention, the inventive robot may comprise at least one nondestructive testing (NDT) device. The NDT device is based on a technique suitable for pipelines, for example magnetic flux leakage, acoustic resonance, ultrasonic or eddy current technique. With these techniques data of the entire circumference of the pipe wall can be provided for structural evaluations. Defects such as pit corrosion and general corrosion, cracks, dents, wrinkles and buckles, coating disbondment, wall thickness and metal loss can be detected.

The NDT device may be, for example, mounted to the robot via a manipulator or robotic arm allowing to move the NDT device within the pipeline. In an embodiment of the present invention the movable arm may carry at least one sensor portion, the sensor portion comprising at least one sensor, the robot being configured to move the arm from a lowered position to a raised position with respect to an upright orientation of the robot to allow the at least one sensor to be positioned substantially coincident with a centreline of a substantially cylindrical pipeline, in use.

In some situations, in the raised position the sensor portion may be positioned so that it is “substantially coincident” with the centreline of the pipeline. The amount by which the sensor portion is raised from the lowered position may be adjusted to accommodate use in pipelines of different respective internal diameters. Therefore the height of the arm may be adjusted so that the sensor portion is “substantially coincident” with the centreline.

The arm may be configured to cause the sensor portion to remain in a substantially fixed orientation with respect to the centreline of the pipeline when the sensor portion is raised and lowered. For example, the robot may be configured such that as the sensor portion is raised or lowered, it experiences translation with respect to the centreline of the pipeline with substantially no relative rotation. In some embodiments the arm may comprise a bar linkage arrangement such as a four bar linkage arrangement allowing raising of the sensor portion without rotation thereof.

The sensor portion may comprise at least one non-destructive testing (NDT) device.

Optionally, said at least one NDT device may comprise a magnetic flux detector, optionally in addition a magnetic flux source. Alternatively or in addition the NDT device may comprise an acoustic detector and optionally an acoustic source. The NDT device may be configured to cause acoustic resonance of a portion of a pipe and to measure the frequency of acoustic resonance. Other arrangements may be useful. Alternatively or in addition the NDT device may comprise an ultrasonic receiver and optionally an ultrasonic transmitter. The NDT device may be arranged to perform ultrasonic inspection. Alternatively or in addition the NDT device may be configured to induce and detect electrical eddy currents in the pipeline. Other arrangements may be useful in some embodiments.

There is at least one sensor on the sensor portion that may comprise a device selected from the group consisting of a temperature sensor, a pressure sensor, a flow sensor and a sensor for sensing the presence of one or more chemical substances. The sensor for sensing chemical substances may be one selected from the group consisting of a methane sensor and a Fourier transform IR (FTIR) spectroscope.

In an embodiment of the invention, the NDT device/manipulator arm is, for example, mounted on top of the robot's body, and the manipulator arm allows moving the NDT device to the center of the pipeline. Of course, the NDT devise may also be mounted underneath or to a side of the robot's body if suitable or desired, and may also be mounted without a manipulator arm.

In yet another embodiment of the invention, the inventive robot may comprise at least one sensor device, for example a temperature sensor, a pressure sensor, a flow sensor or sensors for chemical substances. The sensor for chemical substances may be, for example, a methane sensor for detecting methane or, for more advanced chemical analysis, a Fourier transform IR (FTIR) spectroscope. Of course, also other sensors may be used according to the practical circumstances.

In another embodiment of the invention, the inventive robot may comprise at least one locating device for detecting the position of the robot within the pipeline. The locating device may be, for example, an odometer, a gyro/orientation sensor, an inertial sensor (gyro sensors and accelerometers together) and a global positioning system (GPS) or sonde.

Of course, all optional features of the inventive robot as described above may be combined. Thus, an inventive robot may comprise, for example, a front and rear MV system, a NDT device and any additional sensors needed for specific purposes.

Further, the skilled artisan will appreciate that the inventive robot and/or any MV, NDT or sensor devices may have a modular design. Modular design, or “modularity in design”, is a design approach that subdivides a system into smaller parts called modules, that can be independently created and then used in different systems. A modular system can be characterized by functional partitioning into discrete scalable, reusable modules, rigorous use of well-defined modular interfaces, and making use of industry standards for interfaces. Besides reduction in cost (due to less customization, and shorter learning time), and flexibility in design, modularity offers other benefits such as augmentation (adding new solution by merely plugging in a new module), and exclusion.

If suitable or desired it is also possible to combine two or more inventive robots in a way that one (or more) robots tow or push the others. Of course, the combined robots may be identical or not. Thus, it is possible that, for example, the MV system, the NDT device and any additional sensors are mounted on different robots.

### BRIEF DESCRIPTION OF THE DRAWINGS

One or more embodiments of the invention in its various aspects will now be described, by way of example only, with reference to the accompanying drawings, in which:

FIG. 1 is a plan view schematic illustration of a pipeline inspection robot according to an embodiment of the present invention;

FIG. 2 is a perspective view of the same pipeline inspection robot schematically illustrated in FIG. 1;

FIG. 3 is a perspective view of a magnetic tracked drive means according to an embodiment of the present invention;

FIG. 4 is a perspective view of a magnetic roller means according to an embodiment of the present invention comprising

FIG. 5 is a perspective view of a robotic system according to an embodiment of the invention comprising two robots as defined above, wherein said two robots are connected to each other (here e.g. centre-aligned) in such a way that their at least one tracked drive means and at least one roller means are arranged opposite to each other and when said robotic system travels through a pipeline are efficiently pressed to the pipeline wall.

FIG. 6 is a perspective view showing an inventive robotic system according to another embodiment of the invention comprising a plurality of robots as defined above. The robots are configured to cooperate with one another in such a way that when traveling through a pipeline they are distributed at different respective circumferential locations around the inner bore of the pipeline and move generally parallel to the axis of the pipeline.

### DETAILED DESCRIPTION OF EMBODIMENTS

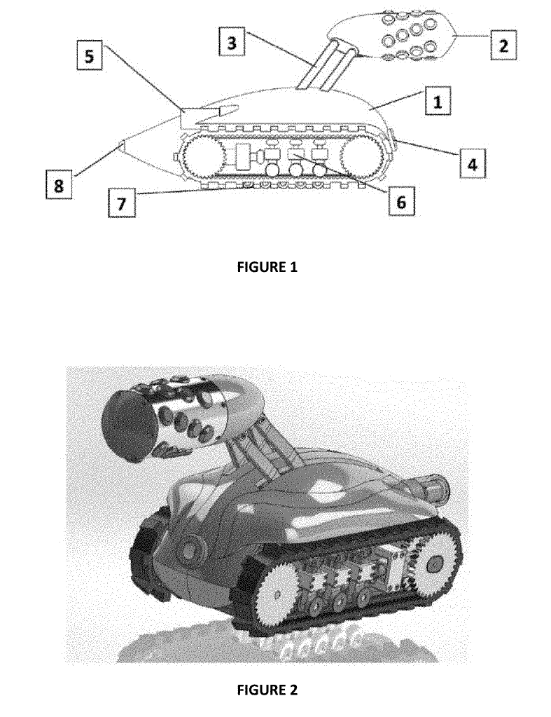

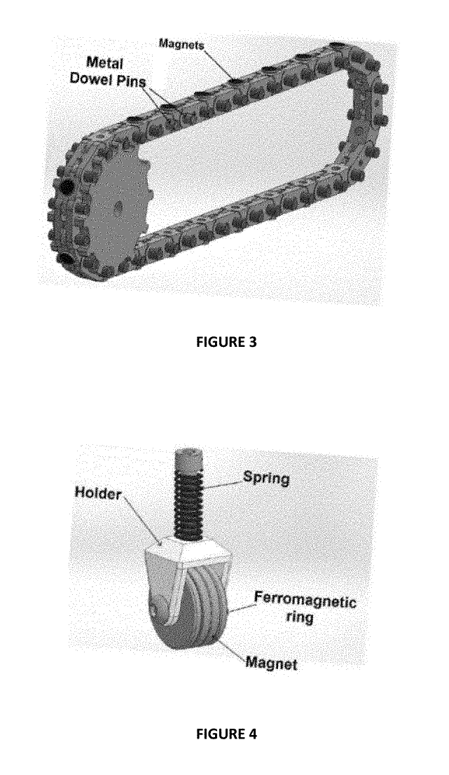

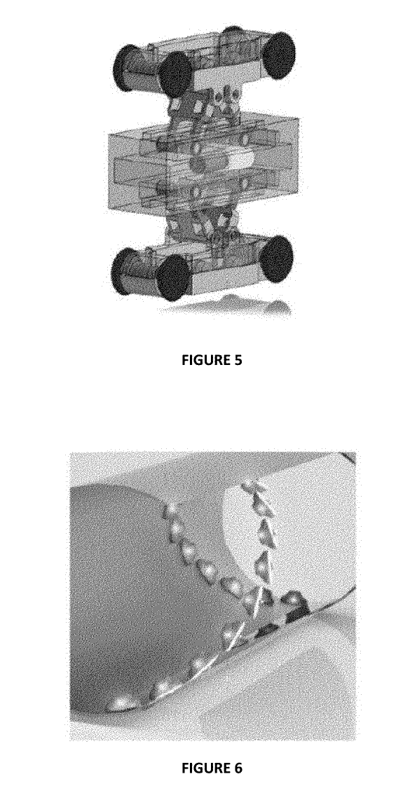

An inventive robot according to one embodiment of the invention is schematically illustrated in FIG. 1. It includes a streamlined aerofoil shaped (low profile) body 1 with NDT articulation arm 3 mounted on a top. By means of the articulation system 3 the modular NDT scanning module 2 can be risen to the centre of pipe (in range of DN600 to DN900). The device drive while monitoring the inside of the pipe by forward-looking field camera 4. During normal operation and reversing the umbilical cable used for data and control signal transmission is observed by rear facing camera 5. Further, the shape of the tracks is adapted to complex pipe geometry and to increase the contact surface area. The suspension system 6 allows the tracks to bend and keep constant traction and required tension. A series of magnetic free rolling wheels 7 increase traction and allows the robot to navigate vertical and incline segment of the pipe. The data and control signals are transmitted through an umbilical cable connected to the robot by a rear connector 8.

In an embodiment of the present invention an umbilical management system (UMS) is used. The UMS may be located inside the launch vessel. Suitable UMSs are commercially available and/or may be easily adapted. For example, a suitable UMS has the following features:
Drum rotation powered by electric motors
Drum rotation manual override for failure mode recovery
Wheeled platform to allow smooth insertion of the UMS into the launch vessel
Motorised umbilical feed to allow the umbilical to be spooled evenly across the drum.
Integrated high-pressure camera (as defined above) to monitor UMS mechanisms and umbilical spooling

1. A robot suitable for travel through a pipeline comprising:
at least one tracked drive means and at least one roller means that can swivel about an axis substantially normal to a rolling axis thereof, wherein said at least one tracked drive means and at least one roller means are provided with magnetic means for generating a magnetic adhesion force between the robot and an internal wall of the pipeline.

2. The robot of claim 1, wherein said robot comprises a body having a streamlined aerofoil shape form that promotes pressing of the robot to the internal wall of the pipeline.

3. (canceled)

4. The robot according to claim 2, wherein said roller means is selected from the group consisting of castor wheels and roller-balls wheels.

5. The robot according to claim, 2, wherein said magnetic means comprises permanent magnets.

6. The robot according to claim 2, wherein said tracked drive means comprises a suspension system allowing the drive tracks to bend and keep constant traction and required tension.

7. The robot according to claim 2, wherein said body is made of an adaptive material which responds to external cues.

8. The robot of claim 2, further comprising at least one machine vision system.

9. The robot according to claim 8, wherein said at least one machine vision system is mounted to the front or rear or to the front and rear of the robot.

10. The robot according to claim 8, wherein said machine vision system comprises at least one video camera selected from the group consisting of a fixed forwards facing camera, a self-levelling camera and a pan and tilt camera.

11. The robot according to claim 10, wherein said at least one video camera operates in the visible or non-visible light spectrum.

12. The robot according to claim 11, wherein said non-visible light spectrum is selected from the group consisting of the infra-red spectrum, the near-infrared spectrum and the ultra-violet spectrum.

13. The robot according to claim 10, wherein said at least one video camera comprises at least one lighting array.

14. The robot according to claim 2, further comprising at least one non-destructive testing (NDT) device.

15. The robot according to claim 14, wherein said NDT device is based on a technique selected from the group consisting of magnetic flux leakage, acoustic resonance, ultrasonic and eddy current.

16. The robot according to claim 14, wherein said NDT device is mounted to said robot via a manipulator arm allowing to move said NDT device within the pipeline.

17. The robot according to claim 2, further comprising at least one sensor device selected from the group consisting of a temperature sensor, a pressure sensor, a flow sensor and sensors for chemical substances.

18. The robot according to claim 17, wherein said sensor for chemical substances is selected from the group consisting of a methane sensor and a Fourier transform IR spectroscope.

19. The robot according to claim 3, further comprising at least one locating device for detecting the position of the robot within the pipeline, the at least one locating device selected from the group consisting of an odometer, a gyro/orientation sensor and a global positioning system (GPS).

20. (canceled)

21. A robotic system comprising two robots, each robot comprising at least one tracked drive means and at least one roller means that can swivel about an axis substantially normal to a rolling axis thereof, wherein said at least one tracked drive means and at least one roller means are provided with magnetic means for generating a magnetic adhesion force between the respective robot and an internal wall of the pipeline, wherein said two robots are either (1) connected to each other in such a way that their at least one tracked drive means and at least one roller means are arranged opposite to each other and when said robotic system travels through a pipeline are efficiently pressed to the pipeline wall; or (2) are configured to cooperate with one another in such a way that when traveling through a pipeline they are distributed at different respective circumferential locations around the inner bore of the pipeline and move generally parallel to the axis of the pipeline.

22. (canceled)

23. A method for pipeline inspection comprising:
moving at least one robot or robotic system along a pipeline within a pipeline network, wherein the at least one robot or robotic system comprises:
at least one tracked drive means and at least one roller means that can swivel about an axis substantially normal to a rolling axis thereof, wherein said at least one tracked drive means and at least one roller means are provided with magnetic means for generating a magnetic adhesion force between the robot and an internal wall of the pipeline;
a body having a streamlined aerofoil shape form that promotes pressing of the robot to the internal wall of the pipeline; and
at least one machine vision system;
inspecting said pipeline for leaks or failures using the at least one machine vison system and/or the at least one NDT device and/or the at least one sensor device of said robot or robotic system; and
tracking the position of said robot or robotic system within said pipeline using the at least one locating device.

24. (canceled)
